(19) United States
(12) Patent Application Publication
(10) Pub. No.: US 2009/0192399 A1
(43) Pub. Date: Jul. 30, 2009

(54) Apparatus and method to detect heart-rate and air conditioning system having the apparatus

(75) Inventors: Hyen Young Choi, Suwon-si (KR); Jeong Su Han, Suwon-si (KR); Su Ho Jo, Seongnam-si (KR); Sung Hoon Kim, Suwon-si (KR); Seong Joo Han, Yongin-si (KR); Sang Jun Lee, Suwon-si (KR); O Do Ryu, Suwon-si (KR)

Correspondence Address: STAAS & HALSEY LLP, SUITE 700, 1201 NEW YORK AVENUE, N.W., WASHINGTON, DC, 20005, US

(73) Assignee: SAMSUNG ELECTRONICS CO., LTD., Suwon-si (KR)

(21) Appl. No.: 12/320,363

(22) Filed: Jan. 23, 2009

(30) Foreign Application Priority Data
Jan. 25, 2008 (KR) 10-2008-8038
Feb. 6, 2008 (KR) 20-2008-1719

(51) Int. Cl. A61B 5/0402; G05D 23/00

(52) U.S. Cl. 600/519; 62/132

(57) ABSTRACT

Disclosed herein are an apparatus and method to detect a heart-rate and an air conditioning system having the apparatus. As peak points of vital signs acquired from a user are detected via determination of a period, a reliable heart rate variability is calculated based on the peak points. As the emotional state of the user can be diagnosed based on vital signs of the user, optimal air-conditioning control according to physical characteristics or emotional state of the user can be accomplished by sequentially controlling a variety of air-conditioning control factors according to the priority thereof until the user becomes comfortable.

### CROSS-REFERENCE TO RELATED APPLICATIONS

This application claims the benefit of Korean Patent Application No. 2008-0008038, filed on Jan. 25, 2008, and Korean Utility Model Application No 2008-0001719, filed on Feb. 5, 2008 in the Korean Intellectual Property Office, the disclosures of which are incorporated herein by reference.

### BACKGROUND

1. Field

The present invention relates to an apparatus and method to detect a heart-rate and an air conditioning system having the apparatus, and, more particularly, to an apparatus and method to effectively detect a Heart Rate Variability (HRV), and an air conditioning system having the heart-rate detecting apparatus.

2. Description of the Related Art

In general, an apparatus to detect a heart-rate is designed to acquire vital signs via a vital sign detecting sensor that comes into contact with a human body, and calculate a heart rate variability from time intervals of peak values of the vital signs. A heart rate variability is used to judge the emotional state of a user using a Fast Fourier Transform (FFT).

In a conventional heart-rate detecting apparatus as disclosed in Korean Patent Laid-Open Publication No. 2003-0081903, after acquiring PhotoPlethysmoGraphy (PPG) signals from a user via a PPG sensor, candidate heart-rate sequences are detected from the PPG signals using a signal processing method based on a wavelet transform and using average heart-rate calculations based on an autocorrelation function. Of the candidate heart-rate sequences, an optimal heart-rate sequence or average heart-rate is extracted.

The above publication also discloses a heart-rate detecting method including: removing high-frequency noise from the PPG signals using the wavelet transform; clipping values above a predetermined level on the basis of a critical value to acquire only signal components related to a heart-rate from the PPG signals having no high-frequency noise; and detecting peak values of the PPG signals via calculation of autocorrelation function values of the clipped signals.

A problem of the above-described conventional detecting method is that using the wavelet transform occupies much memory in signal processing, making it difficult to achieve rapid signal processing and resulting in an excessively long calculation time. Furthermore, clipping of signals using a fixed critical value cannot easily detect accurate peak points because people have different vital sign periods and peak points. This entails deterioration in a probability of detecting an accurate heart rate variability.

### SUMMARY

Therefore, it is an aspect of the present invention to provide an apparatus and method to detect a heart-rate and an air conditioning system having the apparatus, wherein peak points of vital signs of a user are detected using a period detection method rather than a wavelet transform, achieving a reduction in the amount of memory and calculation time and an increased accuracy in the detection of peak points.

It is another aspect of the present invention to provide an apparatus and method to detect a heart-rate and an air conditioning system having the apparatus, wherein, on the basis of the emotional state of a user judged from vital signs of the user, a variety of air-conditioning control factors can be sequentially changed until the user can be comfortable.

Additional aspects and/or advantages will be set forth in part in the description which follows and, in part, will be apparent from the description, or may be learned by practice of the invention.

In accordance with one aspect of the present invention, there is provided a heart rate detecting method including: detecting vital signs of a user; storing the detected vital signs in a unit of a window as a predetermined data size; determining a period of the vital signs using a critical value applied to the window; extracting peak points of the vital signs in the determined period; and calculating a heart rate variability using time information of the extracted peak points.

In accordance with another aspect of the present invention, there is provided a heart rate detecting apparatus including: a vital sign detecting unit to detect vital signs of a user; a storage unit in which the detected vital signs are stored in the unit of a window as a predetermined data size; and a control unit to store the detected vital signs in the storage unit until a data size of the vital signs is equal to the window, to determine a period of the vital signs in the window, to extract peak points of the vital signs based on the determined period, and to calculate a heart rate variability using the extracted peak points.

In accordance with a further aspect of the present invention, there is provided an air conditioning system comprising: a vital sign detecting apparatus to detect vital signs of a user; and an air conditioner to judge an emotional state of the user based on the detected vital signs and to sequentially change a plurality of air-conditioning control factors according to the priority thereof until the user becomes comfortable.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 is a control block diagram of a heart-rate detecting apparatus according to an embodiment of the present invention;

FIG. 2 is a control flow chart illustrating a heart-rate detecting method according to the embodiment of the present invention;

FIG. 3 is a view illustrating vital signs detected in FIG. 2;

FIG. 4 is a view illustrating vital signs detected in FIG. 2, which are stored on a per window basis;

FIG. 5 is a view illustrating determination of a period of vital signs using zero-crossing points in FIG. 2;

FIG. 6 is a view illustrating detection of peak points using a variable critical value in FIG. 2;

FIG. 7 is a configuration view of an air conditioning system according to another embodiment of the present invention;

FIG. 8 is a control block diagram of a heart-rate detecting apparatus shown in FIG. 7;

FIG. 9 is a side sectional view of the air conditioning system shown in FIG. 7;

FIG. 10 is a control block diagram of an air conditioner shown in FIG. 7; and

FIGS. 11A and 11B are a control flow chart illustrating operation of the air conditioning system according to another embodiment of the present invention.

### DETAILED DESCRIPTION OF EMBODIMENTS

Reference will now be made in detail to the embodiments of the present invention, examples of which are illustrated in the accompanying drawings, wherein like reference numerals refer to the like elements throughout. The embodiments are described below to explain the present invention by referring to the figures.

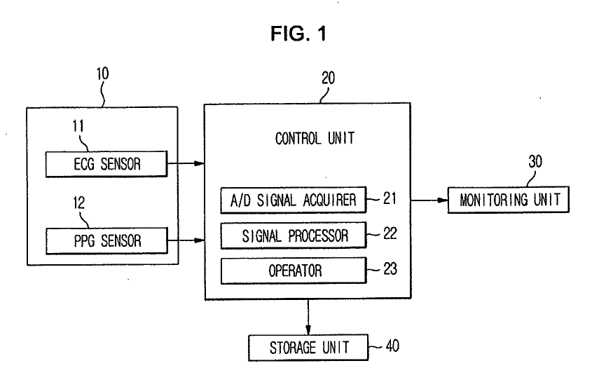

FIG. 1 is a control block diagram of a heart-rate detecting apparatus according to an embodiment of the present invention. As shown in FIG. 1, the heart-rate detecting apparatus according to the present embodiment includes a vital sign detecting unit 10 to detect vital signs from a user, a control unit 20, a monitoring unit 30, and a storage unit 40.

The vital sign detecting unit 10 includes an electrocardiogram (ECG) sensor 11, or photoplethysmography (PPG) sensor 12. The ECG sensor 11 is used to detect ECG signals that represent the electrical activity of a heart during a period of heart beats.

The PPG sensor 12 utilizes the principle in that absorption and reflectivity of light are changed as the diameter of a blood vessel is changed according to a heart rate. The PPG sensor 12 includes a light emitting element to emit infrared light, and a light receiving element to sense light reflected from a user body when the infrared light is directed to the user body. The PPG sensor 12 detects PPG signals as the flow rate of blood is varied depending on a light emission time from the light receiving element.

The control unit 20 includes an A/D signal acquirer 21, a signal processor 22, and an operator 23.

The A/D signal acquirer 21 processes vital signs from the ECG sensor 11 or PPG sensor 12 in an A/D manner. Specifically, the A/D signal acquirer 21 converts vital signs in the form of analog signals into digital signals, enabling acquisition of a greater amount of signals in proportion to a higher sampling frequency.

The signal processor 22 performs a signal processing operation to process the vital signs acquired by the A/D signal acquirer 21 and detect peak points per period.

The operator 23 calculates a heart rate variability from the peak points detected by the signal processor 22.

The control unit 20, having the above-described constituent elements, acquires vital signs, such as ECG signals or PPG signals, via the A/D signal acquirer 21.

The control unit 20 stores the acquired vital signs in the storage unit 40 until a data size of the respective vital signs is equal to a predetermined window size.

Thereafter, the control unit 20 performs a calculating operation to remove, for example, noise from the vital signs via the signal processor 22 and simultaneously performs signal processing operations to determine a period of the vital signals and to detect peak points within the determined period.

Once the peak points within the period are detected, the operator 23 included in the control unit 20 calculates a heart rate variability from time information produced from the peak points.

The control unit 20 sends a series of monitoring results of the vital signs to the monitoring unit 30, to display the results via the monitoring unit 30.

Considering the above-described operation of the control unit 20 in more detail, the control unit 20 senses vital signs, stores the sensed vital signs until a data size of the vital signs is equal to a predetermined window size, and then, determines a period of the vital signs in a window. With relation to determination of the period of the vital signs, more specifically, the control unit 20 first calculates an average value of peak points extracted from a previous window, and sets 1/N^{th} of the calculated average value to a critical value. Then, after detecting zero-crossing points of the vital signs based on the set critical value, the control unit 20 stores time indices of the detected zero-crossing points. Of the stored time indices, a time interval between the neighboring time indices is determined to be a period of the vital signs.

After determining the period of the vital signs, the control unit 20 extracts peak points of the vital signs in the determined period, and calculates a heart rate variability using time information of the extracted peak points. In this case, a value of the determined period is compared with an average period value of the previous window. If the determined period value is more than the average period value, the determined period is judged to be a normal period. If the determined period value is not more than the average period value, the determination of the period is again performed. Under the assumption that the determined period is a normal period, the peak points of the vital signs are extracted in the determined period, and a heart rate variability is calculated using time information of the extracted peak points.

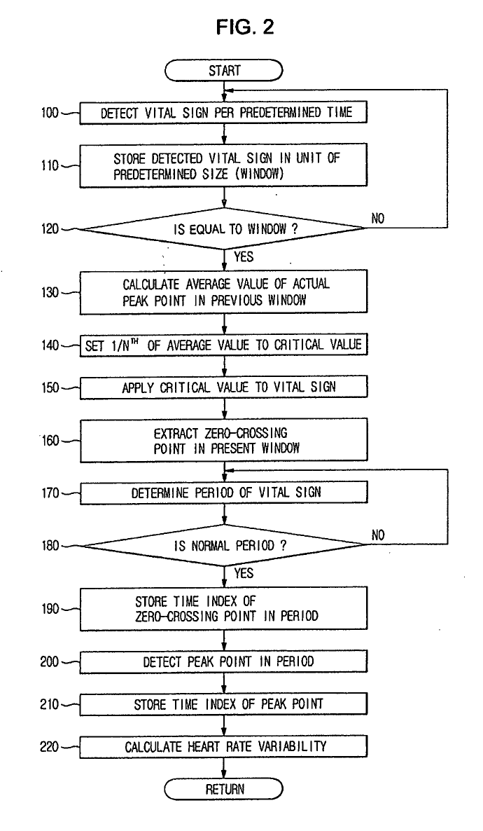

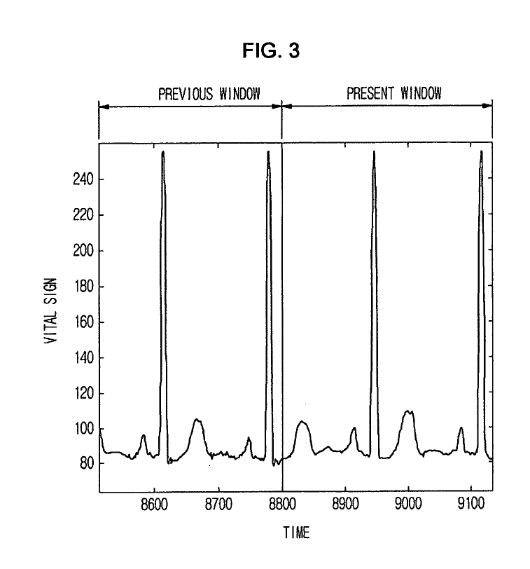

FIG. 2 is a control flow chart illustrating a heart-rate detecting method according to the embodiment of the present invention. Referring to FIG. 2, the control unit 20 detects vital signs per predetermined time (100). When the vital signs are ECG signals, the vital signs take a waveform as shown in FIG. 3.

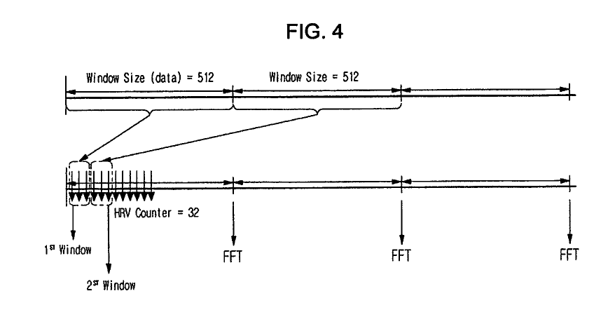

The control unit 20 stores detected vital signs until a data size of the detected vital signs is equal to a predetermined window size (110). If the data size of the detected vital signs is equal to a window size (120), a signal processing operation is performed. As shown in FIG. 4, the control unit 20 acquires vital sign data for each channel every 4 ms. For example, if five-hundred twelve bits of data are acquired via the acquisition of data every 4 ms, a signal processing operation is performed. As will be described hereinafter, peak points are extracted within a single window following the signal processing operation, and a heart rate variability is calculated using an FFT after a total of thirty-two peak points are extracted.

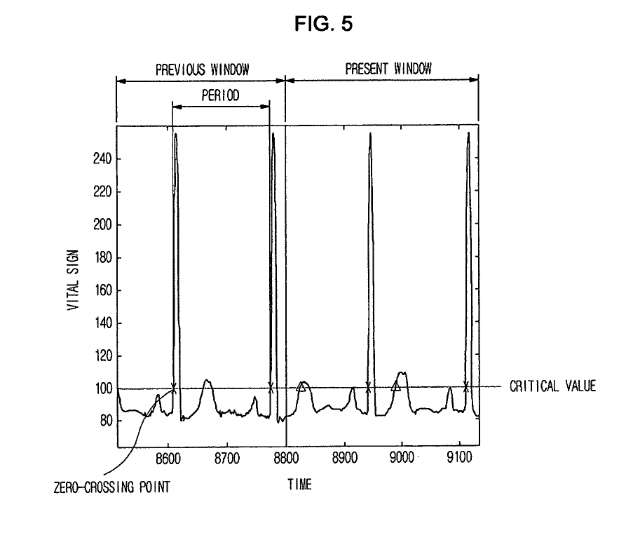

A method to process the acquired data detects a period of the vital signs so as to extract peak points within the period. For this, note that the period of the vital signs must be determined within a single window. As shown in FIG. 5, in the determination of the period, first, a time point, when a sign of the vital signs is changed from negative to positive on the basis of a critical value, is judged to be a zero-crossing point. In particular, when the sign of the vital signs is changed from negative to positive, this situation is judged to be a rising edge. A time interval between the neighboring rising edges based on time indices is determined to be a period. That is, the period is from one rising edge to the next rising edge.

However, as shown in FIG. 5, when the critical value is set low, a plurality of zero-crossing points occur within the single period. Therefore, there is used a method to exclude unavailable zero-crossing points Δ except for zero-crossing points X of a normal period. In this way, the unavailable zero-crossing points Δ accompanied by low peak points can be excluded, and only the available zero-crossing points X can be extracted.

Specifically, in reality, it is necessary to detect only a maximum value within the period by detecting only values above a predetermined numerical value. For this, the critical value is determined using data of a previous window. Values of actual peak points extracted from a previous window are averaged (130), and 1/N^{th} of the average value is set to a critical value (140). In this case, since it is impossible to obtain the average value upon initial control, a preset value is set to the critical value. Here, N is a preset constant.

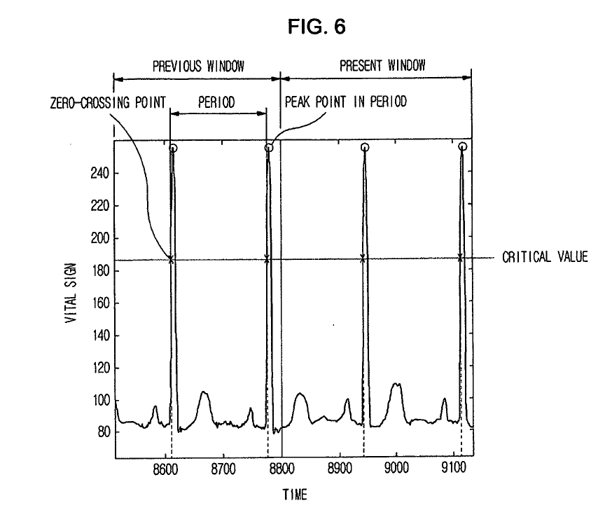

After setting the critical value, the critical value is applied to the vital signs stored in the storage unit 40 (150). That is, if the critical value is applied to the vital signs in such a manner that the critical value is subtracted from the vital signs, the critical value becomes a “zero” level of the vital signs, and unavailable zero-crossing points Δ become values below the “zero” level as shown in FIG. 6. This setting of the critical value is more effective to extract available zero-crossing points X because the critical value is variable based on peak points of a previous window rather than having a fixed value although respective users have different values of peak points of vital signs.

After applying the critical value (160) to the vital signs, zero-crossing points of the vital signs are extracted based on the critical value, and a period of the vital signs is determined using the several zero-crossing points extracted within a present window (170). That is, a difference between time indices of the neighboring zero-crossing points is calculated and determined to be a period.

Once the period is determined, it is judged whether or not the determined period is a normal period (180). Specifically, an average period value of the previous window is calculated, and the calculated average period value is compared with the period value in the present window. The period is determined to be a normal period only when the determined period value is more than an M^{th} of the average period value. Here, M is a preset constant. If the determined period is a normal period, the zero-crossing points are extracted in the period, and time indices of the extracted zero-crossing points are stored in the storage unit 40 (190).

Then, with the use of the stored time indices of the zero-crossing points, a maximum value of the vital signs between a time index [n+1] and a time index [n] is extracted as a peak point within the period (200). A largest value of the vital signs between the last zero-crossing point and the end of the window is stored in memory, and the stored largest value is compared with values of the vital signs until a first zero-crossing point of a next window begins. Thereby, a largest value within the period is extracted as a peak point. Specifically, at a transition time point from the previous window to the present window, a first maximum value from a beginning point of the present window may be erroneously judged to be a peak point in spite of the fact that the peak point does not correspond to a largest value in the present window.

To solve the above-described problem, time indices between the last zero-crossing point of the vital signs in the previous window and the first zero-crossing point of the vital signs in the present window are stored. Then, on the basis of the average peak value of the previous window, only a maximum value above the average peak value in a selected section is extracted as a peak point.

In other cases, data between time indices of the respective zero-crossing points are compared with one another, such that a maximum value is extracted as a peak point.

Thereafter, time indices of the peak points obtained via the above-described signal processing operation are stored in the storage unit 40 (210), and a heart rate variability is calculated using a difference between the neighboring time indices (220). The resulting heart rate variability has a high reliability. Then, the heart rate variability is subjected to frequency analysis via, for example, Fast Fourier Transform (FFT) so as to analyze a power spectrum, such as a strength ratio of a high frequency (HF) band to a low frequency (LF) band. The analyzed result can be used to more accurately diagnose the emotional state of a user so as to determine whether the user is comfortable or uncomfortable.

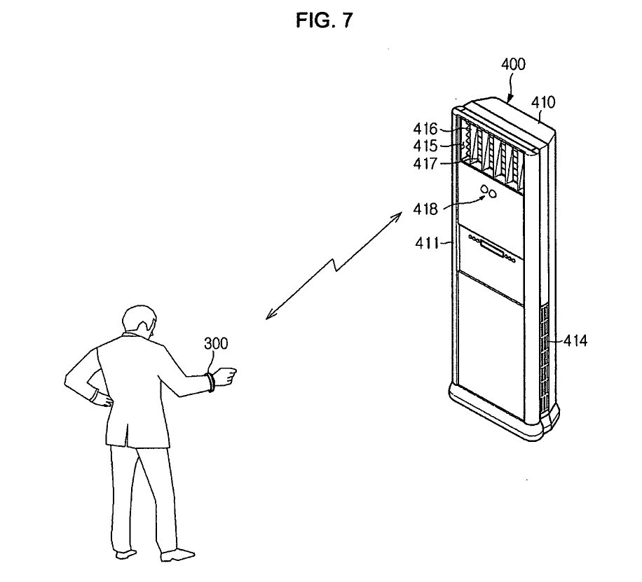

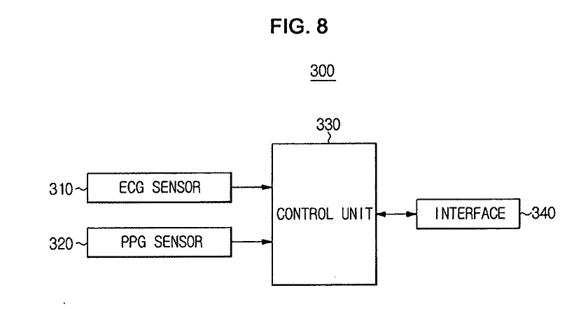

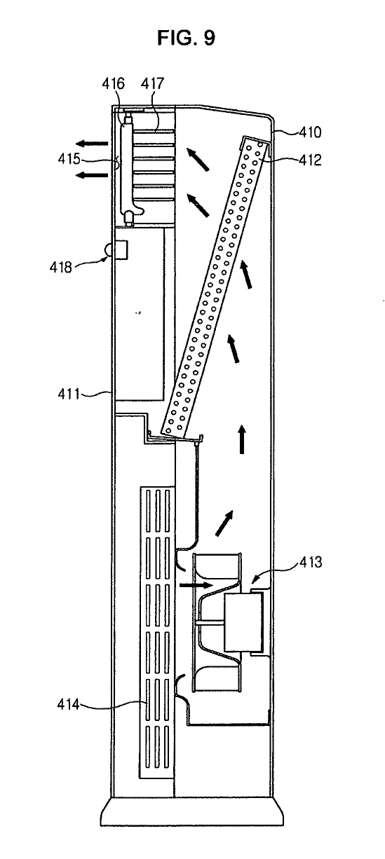

FIG. 7 is a configuration view of an air conditioning system according to another embodiment of the present invention. FIG. 8 is a control block diagram of a heart-rate detecting apparatus shown in FIG. 7. FIG. 9 is a side sectional view of an air conditioner shown in FIG. 7.

As shown in FIGS. 7 to 9, the air conditioning system according to another embodiment includes a vital sign detecting apparatus 300, and an air conditioner 400.

The vital sign detecting apparatus 300 detects vital signs, such as photoplethysmography signals and electrocardiogram signals of a human body, and sends the detected vital signs to the air conditioner 400 in a wireless manner.

The vital sign detecting apparatus 300 includes an electrocardiogram (ECG) sensor 310 to measure the electrocardiogram of a human body or photoplethysmography (PPG) sensor 320 to measure the photoplethysmography of a human body, a control unit 330 to control wireless transmission of the vital signs, such as photoplethysmography signals and electrocardiogram signals, to the air conditioner 400, and an interface 340 to send the vital signs in a wireless manner in communication with the air conditioner 400 according to control signals of the control unit 330.

The ECG sensor 310 includes a plurality of electrodes, which are attached to appropriate positions of a human body and are used to measure and output the electrocardiogram (ECG) of the human body. The electrocardiogram is a waveform representing vital electricity of the electrical activity of the heart during a period of heart beats. If an electric potential of activity is produced during systole and diastole of heart muscles, the electric potential creates current that spreads from the heart to the whole body. The current causes a potential difference according to a position of the body, and the potential difference is vital electricity.

The PPG sensor 320 includes a photoelectric sensor as a combination of infrared diodes and phototransistors and is used to measure and output the photoplethysmogram (PPG) representing a variation in the volume of a peripheral vascular system.

As will be described hereinafter, a Heart Rate Variability (HRV) can be calculated using vital signs, such as ECG and PPG signals. Analyzing the HRV via a power spectrum analysis technique or other analysis techniques enables diagnosis as to whether the emotional state of a user is comfortable or uncomfortable. When a heart rate is continuously varied to maintain homeostasis of a human body against exterior effects, an HRV represents variation of a heart rate per unit time, and the emotional state of a user can be diagnosed from the HRV.

The above-described vital sign detecting apparatus 300 takes the form of a watch band suitable to be worn on and supported by a specific region of the user, for example, the wrist or ankle.

The air conditioner 400 recognizes whether the emotional state of the user is comfortable or uncomfortable on the basis of the vital signs provided from the vital sign detecting apparatus 300. If the user is uncomfortable, the air conditioner 400 performs an optimal air-conditioning control operation according to the physical characteristics or emotional state of the user until the user becomes comfortable. For this, the air conditioner 400 functions to sequentially change a variety of air-conditioning control factors, such as a direction of wind, flow-rate of air, indoor temperature, indoor humidity, etc., according to the priority thereof.

As shown in FIG. 9, the air conditioner 400 includes a box-shaped body 410 having an open front side. A front panel 411 is provided at the open front side of the body 410, to cover the front side of the body 410. A heat exchanger 412 to undergo heat exchange of air and a blower fan 413 to blow air are installed in the body 410.

The body 410 is perforated at lower positions of both lateral sides thereof with first suction holes 414 through which indoor air is suctioned into the body 410. Also, the front panel 411 of the body 410 is perforated at an upper position thereof with a discharge hole 415 through which air, having undergone air-conditioning, is discharged into an indoor space.

A horizontal louver 416 to guide the discharged air horizontally and a vertical louver 417 to guide air vertically are provided inside the discharge hole 415.

A human body sensor 418 is installed below the discharge hole 415 so as to be horizontally pivotally rotated by a predetermined angular range. The human body sensor 418 includes an infrared distance detecting element and an infrared temperature detecting element, and is pivotally rotated by a motor. The human body sensor 418 functions to sense not only a distance from an obstacle disposed in the indoor space, but also a spatial temperature, with respect to the pivoting direction thereof, thereby being capable of sensing the presence of a human body. As will be described hereinafter, the human body sensor 418 is used to control air-conditioning control factors, such as a direction of wind, flow-rate of air, indoor temperature, etc., according to the presence of a human body.

The heat exchanger 412 is arranged in an upper space of the body 410 by a desired inclination to allow air, circulated in the body 410, to undergo heat-exchange while passing through the heat exchanger 412. The blower fan 413 is arranged in a lower space of the body 410 and is used to blow the air, suctioned into the body 410 through the suction holes 414 at both the sides of the body 410, to be directed toward the discharge hole 415 by way of the heat exchanger 412 located above the blower fan 413.

With the above-described configuration of the air conditioner 400, the air, suctioned into the body 410 through the suction holes 414 upon operation of the blower fan 413, undergoes heat exchange while passing through the heat exchanger 412 located in the upper space of the body 410, and thereafter, is again supplied into the indoor space through the discharge hole 415 at the upper position of the body 410.

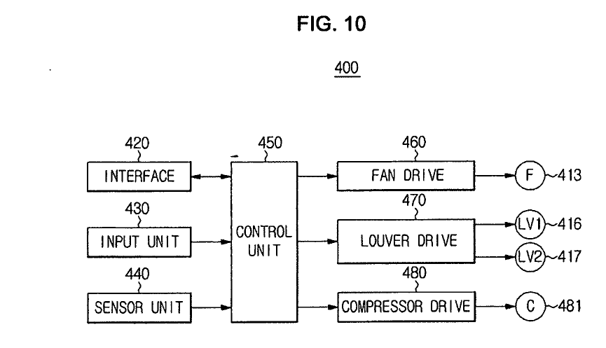

As shown in FIG. 10, the air conditioner 400 of the air conditioning system according to another embodiment of the present invention having the above described configuration further includes a control unit 450.

An interface 420 to send or receive information to or from the vital sign detecting apparatus 300, an input unit 430 to receive a user's command, and a sensor unit 440 including a variety of sensors included in the air conditioner 400 are electrically connected to an input side of the control unit 450.

A fan drive 460 to operate the blower fan 413, a louver drive 470 to operate the horizontal and vertical louvers 416 and 417, and a compressor drive 480 to operate a compressor 481 are electrically connected to an output side of the control unit 450. In addition, the motor to pivotally rotate the human body sensor 418 is electrically connected to the output side of the control unit 450.

The control unit 450 receives vital signs, containing ECG and PPG information, etc., from the vital sign detecting apparatus 300, to calculate an HRV based on a peak interval of the received vital signs, and to analyze a power spectrum via frequency conversion analysis using a Fast Fourier Transform (FFT). The control unit 450 also judges whether the emotional state of the user is comfortable or uncomfortable via the analyzed power spectrum. When the user is uncomfortable, the control unit 450 sequentially changes a variety of air-conditioning control factors according to the priority thereof until the user becomes comfortable, and then, again judges whether or not the user is comfortable. If an affirmative result is obtained, the present control is maintained for a predetermined time. Thereafter, the above-described control operation is continuously performed while judging whether the emotional state of the user is comfortable or uncomfortable. The priority of the variety of air-conditioning control factors may be previously set, or may be manually input via the input unit 430.

Of the variety of air-conditioning control factors, in one example, to control a direction of wind, the louver drive 470 moves the horizontal and vertical louvers 416 and 417, to sequentially control a direction of the air stream. In this case, the control unit 450 first controls the discharge direction of air in a direct-wind mode (or indirect-wind mode) while continuously judging the emotional state of the user and then, if the user is still uncomfortable despite the control operation, the control unit 450 again controls the discharge direction of wind in an indirect-wind mode (or direct-wind mode).

In another example, to control a flow-rate of air, the fan drive 460 regulates a rotating speed of the blower fan 413 so as to sequentially control the flow-rate of air from a strong-wind mode to a weak-wind mode, or vice versa. In this case, the control unit 450 first controls the present flow-rate of air in a strong-wind mode (or weak-wind mode) while continuously judging the emotional state of the user. Then, if the user is still uncomfortable despite the control operation, the control unit 450 again controls the flow-rate of air in a weak-wind mode (or strong-wind mode).

In a further example, to control an indoor temperature, the compressor drive 480 regulates operating efficiency of the compressor 481, so as to sequentially control an indoor temperature from a high-temperature to a low-temperature, or vice versa within a predetermined temperature range. In this case, the control unit 450 first lowers an indoor temperature while continuously judging the emotional state of the user. Then, if the user is still uncomfortable despite the control operation, the control unit 450 raises the indoor temperature.

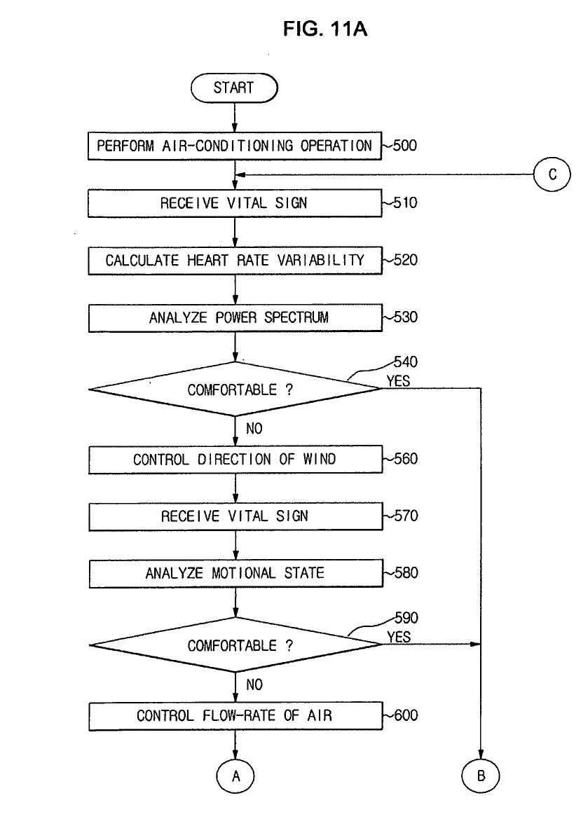

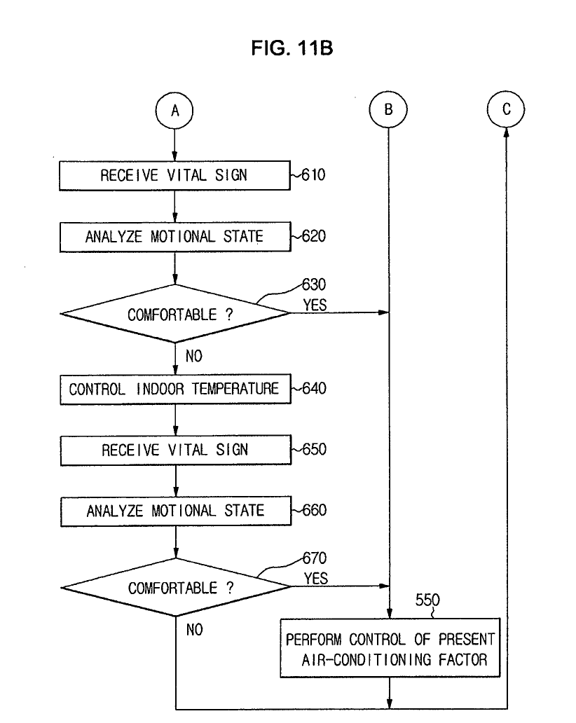

Hereinafter, the above-described operation of the control unit 450 will be described in more detail with reference to FIGS. 11A and 11B. The following description of the present embodiment is limited to the case where there are three air-conditioning control factors including a direction of wind, flow-rate of air, and indoor temperature, and the user determines the priority of the air-conditioning control factors in the sequence of a direction of wind, flow-rate of air, and indoor temperature.

First, the control unit 450 performs an air-conditioning operation wherein suctioned air undergoes air-conditioning and is discharged in a manually or automatically preset direction (500).

After performing the air-conditioning operation, the control unit 450 continuously receives vital signs from the vital sign detecting apparatus 300 for a predetermined time (510), and calculates an HRV using the received vital signs (520).

After calculation of the HRV, the control unit 450 performs certain analysis, such as power spectrum analysis (530), etc. for the HRV, thereby judging whether the emotional state of the user is comfortable or uncomfortable (540). If the user is comfortable, the present air-conditioning control factor is maintained (550).

On the other hand, if the user is uncomfortable, first, a direction of wind is controlled according to the priority of air-conditioning control factors (560). To control a direction of wind, the horizontal and vertical louvers 416 and 417 are moved to sequentially control a direction of wind for a predetermined time, for example, from a direct-wind mode, in which wind is directed toward a person, to an indirect-wind mode in which wind is directed toward a place where no one is present, or vice versa.

During the control of a direction of wind, the control unit 450 again continuously receives the vital signs from the vital sign detecting apparatus 300 for a predetermined time (570), and analyzes the emotional state of the user from the received vital signs (580). Then, on the basis of the analyzed result, the control unit 450 judges the emotional state of the user (590). If the user has become comfortable by virtue of the control of a direction of wind, the present control of a direction of wind is continued for a predetermined time.

On the other hand, if the user is still uncomfortable despite the control of a direction of wind, a flow-rate of air, as the next order following a direction of wind, is controlled (600). Upon control of a flow-rate of air, the rotating speed of the blower fan 413 is controlled to sequentially control a flow-rate of discharged air from a strong-wind mode to a weak-wind mode, or vice versa.

During control of a flow-rate of air, the control unit 450 again continuously receives the vital signs from the vital sign detecting apparatus 300 for a predetermined time (610), and analyzes the emotional state of the user from the received vital signs (620). Then, on the basis of the analyzed result, the control unit 450 judges the emotional state of the user (630). If the user has become comfortable by virtue of control of a flow-rate of wind, the present control of a flow-rate of air is continued for a predetermined time.

On the other hand, if the user is still uncomfortable despite the control of a flow-rate of air, an indoor temperature, as the next order following a flow-rate of air, is controlled (640). Upon control of an indoor temperature, the operating efficiency of the compressor 481 is controlled to sequentially control an indoor temperature within a predetermined temperature range, so as to lower or raise an indoor temperature.

During control of an indoor temperature, the control unit 450 again continuously receives the vital signs from the vital sign detecting apparatus 300 for a predetermined time (650), and analyzes the emotional state of the user from the received vital signs (660). Then, on the basis of the analyzed result, the control unit 450 judges the emotional state of the user (670). If the user has become comfortable by virtue of control of an indoor temperature, the present control of an indoor temperature is continued for a predetermined time.

On the other hand, if the user is still uncomfortable despite the control of an indoor temperature, the control unit 450 returns to the operation 510, continuously performing the following operations.

Although the embodiments of the present embodiment explain individual control of the air-conditioning control factors including a direction of wind, flow-rate of air and indoor temperature, the embodiments of the present invention are not limited thereto, and two or more air-conditioning control factors may be controlled together. This is possible as the user selects any one of various combinations of air-conditioning control factors upon determination of the priority of the factors.

As is apparent from the above description, in a heart-rate detecting method according to the present embodiments, as peak points of vital signs acquired from a user are detected via determination of a period, a reliable heart rate variability can be calculated based on the peak points. As compared to a conventional method wherein peak points of vital signs are detected using a wavelet transform, the detecting method according to the embodiments of the present invention can reduce memory usage and calculation time, and exhibit a faster response to a variation of vital signs while assuring more rapid and accurate detection of the peak points.

In accordance with another embodiment, as the emotional state of the user can be diagnosed based on vital signs of the user, optimal air-conditioning control according to physical characteristics or emotional state of the user can be accomplished by sequentially controlling a variety of air-conditioning control factors according to the priority thereof until the user becomes comfortable.

Although a few embodiments have been shown and described, it would be appreciated by those skilled in the art that changes may be made in these embodiments without departing from the principles and spirit of the invention, the scope of which is defined in the claims and their equivalents.

What is claimed is:

1. A heart rate detecting method comprising:
detecting vital signs of a user;
storing the detected vital signs in a unit of a window as a predetermined data size;
determining a period of the vital signs using a critical value applied to the window;
extracting peak points of the vital signs in the determined period; and
calculating a heart rate variability using time information of the extracted peak points.

2. The method according to claim 1, wherein the determining of the period includes:
setting the critical value;
extracting zero-crossing points of the vital signs on the basis of the set critical value in the window; and
determining the period of the vital signs based on time indices of the extracted zero-crossing points.

3. The method according to claim 2, wherein the setting of the critical value includes:
calculating an average value of peak points sensed in a previous window; and
setting 1/N^{th} of the calculated average value to the critical value.

4. The method according to claim 2, wherein the zero-crossing points have a zero level corresponding to the critical value.

5. The method according to claim 2, further comprising:
determining whether or not the determined period is a normal period.

6. The method according to claim 5, wherein the determining of the normal period includes:
comparing a value of the determined period with an average period value of a previous window; and
determining the determined period is the normal period if the determined period value is larger than or equal to the average period value.

7. The method according to claim 6, wherein, if the determined period is the normal period, the extracting of peak points includes extracting peak points of the vital signs corresponding to a maximum value within the determined period.

8. The method according to claim 7, wherein, at a transition time point from the previous window to a present window, if a maximum value between a last zero-crossing point of the previous window and a first zero-crossing point of the present window is more than an average value of peak points in the previous window, the extraction of peak points includes extracting peak points corresponding to the maximum value.

9. A heart rate detecting apparatus comprising:
a vital sign detecting unit to detect vital signs of a user;
a storage unit in which the detected vital signs are stored in the unit of a window as a predetermined data size; and
a control unit to store the detected vital signs in the storage unit until a data size of the vital signs is equal to the window, to determine a period of the vital signs in the window, to extract peak points of the vital signs based on the determined period, and to calculate a heart rate variability using the extracted peak points.

10. The apparatus according to claim 9, wherein the control unit calculates an average value of peak points extracted from a previous window, to set 1/N^{th} of the calculated average value to a critical value, to extract zero-crossing points of the vital signs on the basis of the set critical value, to store time indices of the extracted zero-crossing points, and to determine a period of the vital signs from a time interval between the stored neighboring time indices.

11. The apparatus according to claim 9, wherein the control unit compares a value of the determined period with an average period value of a previous window, and determines that the determined period is a normal period if the determined period value is larger than or equal to the average period value.

12. The apparatus according to claim 11, wherein the control unit extracts peak points of the vital signs corresponding to a maximum value within the determined period when the determined period is a normal period.

13. The apparatus according to claim 9, wherein, at a transition time point from a previous window to a present window, if a maximum value between a last zero-crossing point of the previous window and a first zero-crossing point of the present window is more than an average value of peak points in the previous window, the control unit extracts peak points corresponding to the maximum value.

14. An air conditioning system comprising:
a vital sign detecting apparatus to detect vital signs of a user; and
an air conditioner to judge an emotional state of the user based on the detected vital signs and to sequentially change a plurality of air-conditioning control factors according to the priority thereof until the user becomes comfortable.

15. The system according to claim 14, wherein the plurality of air-conditioning factors include at least two of a direction of air, a flow-rate of the air, and an indoor temperature.

16. The system according to claim 14, wherein the priority is input by the user.

17. The system according to claim 14, wherein the vital sign detecting apparatus contacts a body of the user, to detect the vital signs.
